(19) United States
(12) Patent Application Publication
(10) Pub. No.: US 2014/0107524 A1
(43) Pub. Date: Apr. 17, 2014

(54) NEUROMUSCULAR MONITORING DISPLAY SYSTEM

(71) Applicants: Mayo Foundation for Medical Education and Research, Rochester, MN (US); T4 Analytics LLC, Dover, DE (US)

(72) Inventors: Sorin Joseph Brull, Ponte Vedra Beach, FL (US); David Robert Hampton, Woodinville, WA (US); Jacoba Alberta Witteveen, Maastricht (NL)

(73) Assignees: Mayo Foundation for Medical Education and Research, Rochester, MN (US); T4 Analytics LLC, Dover, DE (US)

(21) Appl. No.: 14/051,785

(22) Filed: Oct. 11, 2013

Related U.S. Application Data
(60) Provisional application No. 61/713,202, filed on Oct. 12, 2012.

(51) Int. Cl. A61B 5/04; A61B 5/00

(52) U.S. Cl. CPC A61B 5/04001; A61B 5/742
USPC 600/554

(57) ABSTRACT

Disclosed herein is a system for displaying a degree of neuromuscular block in a patient. An example system can include: a display unit having a graphical user interface (GUI); a processor; and a memory. The system can be configured to: receive data in response to a pattern of stimuli applied to the patient according to a stimulation protocol; determine the degree of neuromuscular block based on the received data; display a numerical representation corresponding to the degree of neuromuscular block; display a graphical representation corresponding to the degree of neuromuscular block and display a timer related to the stimulation protocol. The numerical and graphical representations can be displayed in first and second regions of the GUI, respectively. Additionally, a display color of at least a portion of the first region, the numerical and graphical representations can be configured to dynamically change based on the degree of neuromuscular block.

### CROSS-REFERENCE TO RELATED APPLICATIONS

This application claims the benefit of U.S. Provisional Patent Application No. 61/713,202, filed on Oct. 12, 2012, entitled “NEUROMUSCULAR MONITORING DISPLAY SYSTEM,” the disclosure of which is expressly incorporated herein by reference in its entirety.

### BACKGROUND

About 230 million surgeries take place annually world-wide; 40 million US patients undergo in-hospital general anesthesia, which induces loss of consciousness, each year, and 25 million of those also receive muscle relaxants (also called neuromuscular blocking agents, NMBAs), which inhibit neuromuscular transmission. These relaxant agents decrease muscle tension and suppress reflex contractions, and may be administered for several reasons including the following.

Muscle relaxants (NMBAs) have two forms: depolarizing agents, which are short-acting (5-10 min duration) and are sometimes used at the start of anesthesia to facilitate tracheal intubation, and non-depolarizing agents that have a longer duration of action (20-60 min), and that are used to maintain muscle relaxation during surgery. The effects of non-depolarizing agents start within minutes and continue for up to 20-60 minutes after withdrawal (depending on the type of relaxant used), so they must be administered repeatedly throughout the surgical procedure.

Drug effects must completely dissipate once the surgical procedure is complete, however, so that patients can start breathing on their own (spontaneously). Reversal drugs (anticholinesterases) can be administered to speed-up recovery from muscle relaxants, but reversal drugs can slow the heart to dangerous levels (bradycardia), and can have a host of other unpleasant side effects, such that atropine (or glycopyrrolate) is commonly administered as an adjunct to reversal agents. Unfortunately, atropine and atropine-like agents also have their own additional side-effects, such as nausea, vomiting and tachycardia.

Overdosing of relaxants to assure complete muscle paralysis during surgery can lead to delayed recovery of muscle function, prolonging recovery room stays, hospital stays and increasing healthcare costs. 30-60% of patients admitted to the postoperative care unit (Recovery Room, or PACU) have significant residual muscle weakness (i.e., incomplete reversal of paralysis). In extreme cases, patients can experience a Critical Respiratory Event (CRE) in which they are unable to breathe independently. CRE affects 0.8% of patients who have residual weakness, and may require emergency placement of another breathing tube; approximately 10,000 patients are estimated to require emergent re-insertion of the breathing tube each year from complications of post-surgical CRE. The need for emergent reintubation leads to morbidity and mortality, and markedly increases the cost of healthcare.

An optimal dose of paralytic (muscle relaxant) medications should be based on the effect that they have on muscles, rather than dosing based on physical characteristics of the patient (age, sex, weight) or drug concentration (blood or tissue). Unfortunately, simple subjective assessment of muscle tone, spontaneous breathing, and reflex responses are not accurate or consistent indicators of relaxant effect. Neuromuscular monitoring systems have been proposed to give more precise indication of the degree of neuromuscular block.

### SUMMARY

Disclosed herein is a system (e.g., a neuromuscular monitoring system) for displaying a degree of neuromuscular block in a patient. In particular, a system including a graphical user interface (GUI) for intuitively presenting the degree of neuromuscular block in the patient is disclosed. For example, a system for displaying a degree of neuromuscular block in a patient can include: a display unit having a GUI; a processor; and a memory coupled to the processor. The memory can have computer-executable instructions stored thereon that, when executed by the processor, cause the system to: receive data in response to a pattern of one or more stimuli applied to the patient according to a stimulation protocol; determine the degree of neuromuscular block in the patient based on the received data; display a numerical representation corresponding to the degree of neuromuscular block in the patient; display a graphical representation corresponding to the degree of neuromuscular block in the patient and display a timer related to the stimulation protocol on the GUI. According to some implementations, the numerical representation can be displayed in a first region of the GUI, and the graphical representation can be displayed in a second region of the GUI. Additionally, a display color of at least a portion of the first region, the numerical representation and the graphical representation can be configured to dynamically change based on the degree of neuromuscular block in the patient.

In some implementations, the numerical representation can be a ratio, a percentage or a count of each non-zero electrical response of a muscle to the pattern of one or more stimuli, the ratio, percentage or the count being related to the degree of neuromuscular block in the patient. Alternatively or additionally, the graphical representation can depict an electrical response of a muscle to the pattern of one or more stimuli applied to the patient according to the stimulation protocol.

Additionally, the display color can be a first color when the numerical representation is greater than or equal to a first predetermined value. The display color can be a second color when the numerical representation is greater than or equal to a second predetermined value and less than the first predetermined value. The display color can be a third color when the numerical representation is less than the second predetermined value. In some implementations, the first predetermined value can be 0.9 or 90% and the second predetermined value can be 0.4 or 40%. In addition, the first, second and third colors can be green, yellow and red, respectively.

In some implementations, the first region of the GUI can define a closed-loop shape, and the at least a portion of the first region can extend adjacent to at least a portion of a perimeter of the closed-loop shape. For example, the closed-loop shape can be at least one of a circle or a polygon.

Additionally, the timer can be a graphical timer that extends adjacent to at least a portion of the perimeter of the closed-loop shape. The graphical timer can depict a time between successive applications of the pattern of one or more stimuli applied to the patient according to the stimulation protocol, for example. In some implementations, the memory can have further computer-executable instructions stored thereon that, when executed by the processor, cause the system to dynamically change the graphical timer in a clockwise or counterclockwise direction.

In some implementations, the pattern of one or more stimuli comprises a plurality of stimuli, each stimulus being applied after a predetermined time interval. Additionally, the memory can have further computer-executable instructions stored thereon that, when executed by the processor, cause the system to record an electrical response of a muscle to each of the plurality of stimuli.

Optionally, the graphical representation can be an amplitude of the electrical response of the muscle to each of the plurality of stimuli.

Alternatively or additionally, the graphical representation can be a ratio of an amplitude of the electrical response of the muscle to each of the plurality of stimuli to a control amplitude. In some implementations, the control amplitude can be an amplitude of the electrical response of the muscle to one of the plurality of stimuli applied at approximately a beginning of the stimulation protocol. Optionally, the control amplitude can be an amplitude of the electrical response of the muscle to a first stimulus of the plurality of stimuli.

In some implementations, the plurality of stimuli can include at least four stimuli. In this case, the graphical representation can be a ratio of an amplitude of each of a plurality of subsequently applied stimuli of the plurality of stimuli to an amplitude of the electrical response of the muscle to a prior stimulus of the plurality of stimuli. For example, the graphical representation can be a ratio of an amplitude of the electrical response of the muscle to a second, third and fourth stimulus of the plurality of stimuli to an amplitude of the electrical response of the muscle to a first stimulus of the plurality of stimuli. Or, the graphical representation can be a ratio of an amplitude of the electrical response of the muscle to a fifth or greater stimulus of the plurality of stimuli to an amplitude of the electrical response of the muscle to a first stimulus of the plurality of stimuli. Alternatively or additionally, the stimulation protocol is a train-of-four protocol, a train-of-four count protocol, a tetanic protocol or a post-tetanic count protocol.

In some implementations, the first region of the GUI can define a circle when the protocol is a train-of-four protocol or a train-of-four count protocol. In other implementations, the first region of the GUI can define a polygon when the protocol is a tetanic protocol or a post-tetanic count protocol. For example, the polygon can be a triangle.

Alternatively or additionally, the numerical representation can optionally be a count of each non-zero electrical response of the muscle to the plurality of stimuli. In these implementations, the numerical representation can be related to the degree of neuromuscular block in the patient.

Optionally, the memory can have further computer-executable instructions stored thereon that, when executed by the processor, cause the system to display at least one icon on the GUI. For example, the icon can indicate at least one of a battery charge, the patient's skin temperature or a system status.

Alternatively or additionally, the memory can have further computer-executable instructions stored thereon that, when executed by the processor, cause the system to display a menu bar on the GUI.

It should be understood that the above-described subject matter may also be implemented as a computer-implemented method, a computer process, or an article of manufacture, such as a tangible computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates an example GUI according to an implementation discussed herein;

FIGS. 2A-2C illustrate example GUIs according to implementations discussed herein;

FIG. 2D illustrates an example of a first region of a GUI defining a polygon according to an implementation discussed herein;

FIGS. 3A-3C illustrate example GUIs according to implementations discussed herein; and

FIG. 4 is a block diagram of a computing device according to an implementation discussed herein.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms. While implementations will be described for displaying a degree of neuromuscular block in a patient, it will become evident to those skilled in the art that the implementations are not limited thereto.

A neuromuscular monitoring system can optionally be used to assess neuromuscular blockade in a subject who has received a muscle relaxant agent. The muscle relaxant agent is optionally a neuromuscular blocking agent. Optionally, the muscle relaxant agent is a depolarizing agent. Optionally, the muscle relaxant agent is a non-depolarizing agent.

The neuromuscular monitoring system provides an objective measure of nerve and muscle function that corresponds directly to effects that the muscle relaxant agent has on the body. Relaxants can thus be more effectively administered and reversed, providing more precise control over induction of anesthesia and relaxation, and identifying when surgical procedures can be started safely. Periodic muscle function monitoring can also guide the titration of muscle relaxants during the surgery to avoid over- and under-dosing, and can signal when a patient has adequately responded so that the endotracheal (breathing) tube can be introduced (at the beginning of the surgical procedure) or withdrawn (at the end of surgical procedure).

The neuromuscular monitoring system can optionally be used to objectively measure the depth of neuromuscular blockade accurately and continuously throughout surgical procedures. The neuromuscular function is directly assessed by comparing the evoked muscle response (the evoked electrical activity behind the muscle “twitch”) in response to electrical stimulation of the corresponding motor nerve. Adequate muscle relaxation has been achieved when the muscle response to repetitive stimulation is extinguished while nerve conduction remains intact. The neuromuscular monitoring system repeats the assessment when manually or automatically triggered (at user-selected intervals), providing ongoing monitoring of neuromuscular function status throughout any procedure, using any peripheral motor nerve.

As discussed above, muscle relaxants are administered during some types of surgeries. Muscle relaxants interrupt the chemical conduction across the neuromuscular junction, but do not affect the electrical conduction in either the nerve or the muscle fibers. In particular, the muscle relaxants block receptor sites, which prevent chemical messengers from initiating an electrical response in the muscle fiber. As more receptor sites are blocked, fewer muscle fibers receive stimulation, and both the visible mechanical twitch and the underlying electrical response in the muscle decrease. A single administration of muscle relaxants causes a rapid decrease in the response of the muscle, which then restores to normal over time as the drug is metabolized and then excreted by the body (spontaneous recovery). For a particular dose of muscle relaxant, the magnitude of decrease of the muscle response depends on both the time since drug administration and the muscle that is being monitored. For example, the thumb muscle is affected to a greater degree for the same dose of muscle relaxants than the diaphragm. Successful monitoring, therefore, depends both on identifying the correct muscle, and on continuous monitoring of the evolving effect of muscle relaxant administration and withdrawal (reversal).

Prior to administering the muscle relaxants to the patient, a nerve impulse evoked by the stimulation travels to the muscle and elicits an electrical response that results in a muscle twitch. As the muscle relaxants are applied, the receptor sites are blocked and only some muscle fibers respond. Thus, although the nerve response remains unchanged in strength, the amplitude of the muscle response diminishes, an effect more pronounced in the muscle twitch than in the electrical recording. At full block, all muscle responses are abolished, but the nerve response is preserved. Thus, it is possible to detect a procedural error in the case where the stimulus is moved distant to the nerve, because in such a case, there will be neither a detected nerve response nor a muscle response (twitch).

An example neuromuscular monitoring system can include a stimulating/recording unit and a control/visualization unit. The stimulating/recording unit may include a nerve stimulator and sensors (e.g., recording electrodes or sensing electrodes). The nerve stimulator is capable of delivering electrical pulses to a motor nerve such as the median or ulnar nerve at the wrist, the tibial nerve at the ankle or the facial nerve beneath the ear, for example. For example, the nerve stimulator may deliver a 200 μs or 300 μs square-wave, monophasic, constant electrical pulse. The electrical pulse delivered by the nerve stimulator should be sufficient in strength to elicit nerve responses when the patient is in an unblocked state. In addition, the nerve stimulator may be capable of delivering sequences of pulses, for example train-of-four (TOF) and tetanic bursts.

The sensors are capable of sensing the intrinsic electrical activity of the nerve and muscle, which are induced by the nerve stimulation. By sensing the electrical activity of the muscle, for example, it is possible to measure the amplitude of the electrical activity, which directly corresponds to the strength of the muscle response. Accordingly, it is possible to determine the impact that the muscle relaxants have on the patient at any point in time during the surgery because changes in the amplitude of the electrical activity of the muscle can be correlated directly to changes caused by addition and reversal of the muscle relaxants.

The control/visualization unit may contain user-input controls and a visual display, store operating protocols, collect patient data and generate a system clock. For example, the control/visualization unit may include input and output devices, a processing device, an IV-pole holder and an external communication link. The input and output devices may include user-input controls such as, for example, a power on/off control, a test protocol selection control (single twitch, TOF, tetanic, Post-tetanic count (PTC)), a stimulus intensity control (0-100 mA constant current), a stimulus mode control (manual or continuous), a stimulus trigger control, etc. The user-input controls may consist of backlit buttons for indicating active modes and successful selections, and audible tones may optionally be used for alarms. In addition, the user-input controls may be designed such that the user can operate the controls while wearing surgical gloves. The visual display may be capable of displaying a visual indicator that the control/visualization unit is on, fault indicators (i.e., low battery, loss of electric continuity, failure to deliver stimulus, loss of communication connection), stimulus intensity, bar graphs representing responses to the stimuli, etc. The visual display is discussed in detail below.

The degree of neuromuscular block in the patient can be determined according to a stimulation protocol. For example, the stimulation protocol can include applying a pattern of one or more stimuli to the patient, recording for the nerve and/or muscle response (e.g., the electrical activity of the stimulated nerve and/or innervated muscle) and determining the degree of neuromuscular block in the patient based on the recorded response. The TOF protocol is one example stimulation protocol. The TOF protocol consists of applying a predetermined pattern of stimuli at predetermined intervals to the motor nerve. The stimulus may be a 200 μs or 300 μs, square-wave, monophasic, fixed width between 100 μs and 300 μs constant current electrical pulse. Optionally, the stimulus duration may be longer or shorter than 200 μs, including but not limited to a duration between 100 and 300 μs. The nerve and muscle responses are recorded by the sensing electrodes. The predetermined number is preferably four stimuli, but it may also be five, six, seven, etc.

After recording the nerve and muscle responses, the amplitude of the muscle response is measured. The amplitude may be the peak-to-peak or the baseline-to-peak amplitude. The measured amplitude may be compared to a control amplitude to determine the level of neuromuscular block. For example, the control amplitude may be zero. When the predetermined pattern of stimuli is applied to the patient before administration of the muscle relaxants, the amplitude of the muscle responses are expected to be approximately equal and non-zero. However, as muscle relaxants are administered to the patient, the amplitude of each subsequent muscle response diminishes. In one implementation, the amplitude decreases to zero, preferably by the fourth recorded muscle response, which may indicate a certain degree of neuromuscular block.

Additionally, the TOF ratio may be determined by calculating a ratio of amplitudes of any two, distinct muscle responses to a train of sequentially applied stimuli. In some implementations, the ratio may be a ratio of the amplitude of a subsequent muscle response (i.e., recorded later in time) to the amplitude of a previous muscle response (i.e., recorded earlier in time). For example, the train-of-four ratio is the ratio of the amplitude of the fourth sequentially applied stimulus to the first sequentially applied stimulus in a train of sequentially applied stimuli. The TOF ratio may then be compared to a control ratio (which should preferably be 1.0). Preferably, the TOF ratio will be a ratio of the amplitude of the fourth muscle response to the amplitude of the first muscle response, but can alternatively be the ratio of the amplitudes of any of the first, second, third, fourth, fifth, six, etc. muscle responses. In an unblocked state, the TOF ratio is approximately 1.0. As the neuromuscular block deepens, the TOF ratio falls progressively to 0.0. Thus, a smaller TOF ratio, i.e., one that approaches 0.0, corresponds to a greater level of neuromuscular block, and a TOF ratio of the fourth to the first muscle response of 0.0 indicates approximately greater than or equal to 80% neuromuscular block (receptor occupancy).

In addition, it is possible to determine the TOF count according to a TOF count protocol. For example, when the TOF ratio is 0.0 (i.e., the fourth muscle response is non-existent), a determination is made as to how many stimuli (i.e., first, second and third stimuli) exhibited a non-zero response. As neuromuscular block deepens, the TOF count decreases from three counts to zero. For example, when the TOF ratio is 0.0 and the TOF count is zero, the neuromuscular block is approximately greater than or equal to 95%. In contrast, as neuromuscular block lessens, the TOF count increases. When the TOF ratio is 0.9 (and the TOF count is, by definition, four), the neuromuscular block is approximately less than or equal to 70%. This level of neuromuscular function (less than 70% block) is considered the threshold for adequate recovery. This disclosure contemplates that the TOF count may be calculated for greater than four applied stimuli.

Another example stimulation protocol is the tetanic protocol. Similarly to the TOF protocol, the tetanic protocol consists of a predetermined pattern of stimuli applied at predetermined intervals. Unlike the TOF protocol, however, the tetanic protocol consists of applying a larger number of stimuli at a higher frequency. The stimuli can be applied at a frequency greater than 30 Hz (e.g., between 50 Hz and 100 Hz, for example). For example, 250 or 500 electrical pulses may be applied at a rate of 50 or 100 Hz in a five-second period. In addition, each stimulus (electrical pulse) may have a duration of 200 μs, or, optionally, a duration greater than or less than 200 μs. The nerve and muscle responses are recorded by the sensing electrodes.

After recording the nerve and muscle responses, the amplitude of the muscle responses is measured, and the tetanic ratio is calculated. Similarly to the TOF ratio, the tetanic ratio may be the ratio of an amplitude of a subsequently applied stimulus (or series of stimuli) to an amplitude of a previously applied stimulus (or series of stimuli), i.e., the last stimulus to the first stimulus in the train of stimuli (or a combination of later-in-time series of stimuli to earlier-in-time series of stimuli). Because there may be some amplitude variation in the evoked muscle responses at the beginning of the tetanic stimulation, a ratio of the amplitude of any response toward the end of the stimulation to the amplitude of any response toward the beginning of the stimulation may be calculated, and a value less than 1.0 demonstrates the presence of neuromuscular block. For example, there may be some amplitude variation in the evoked responses during the first 1-3 seconds of the stimulation. In some implementations, the response towards the beginning of the stimulation with the largest amplitude may be used in the ratio. However, as discussed above, the ratio may be the ratio of amplitudes of any two, distinct muscle responses to a train of sequentially applied stimuli. As the neuromuscular block deepens, the tetanic ratio falls progressively from a normal baseline of 1.0 towards 0.0. Thus, a smaller tetanic ratio, i.e., one that approaches 0.0, corresponds to a greater level of neuromuscular block. If the tetanic ratio equals zero, the tetanic duration may be calculated. The tetanic duration may be calculated by estimating the duration of the time interval between the non-zero start and the end of the response, i.e., 0.1-4.9 seconds. As discussed above, during normal neuromuscular transmission, the evoked muscle responses to the tetanic stimulation merge into a single sustained contraction of the muscle. However, during neuromuscular block, the amplitude of responses to the tetanic stimulation will not be sustained (i.e., fade occurs). Accordingly, the level of neuromuscular block may correspond to the time interval of the response.

In addition, it is possible to determine the post-tetanic count (PTC). When a deep neuromuscular block is achieved, and estimation using either the TOF protocol or the tetanic protocol is not elicited, it may be possible to elicit a response using a special stimulus protocol, i.e., the PTC protocol. The PTC protocol includes a first According to the PTC protocol, the first stimulus is a tetanic stimulation, or a pattern of 250 or 500 stimuli (each of 200 μs duration) applied at, optionally, 50 or 100 Hz during a five-second period. Optionally, the duration of each stimulus may be longer or shorter than 200 μs. The nerve and muscle responses are recorded using the sensing electrodes. After expiration of a predetermined time interval (e.g., 20-30 seconds) from application of the first stimulus, a second stimulus is applied. For example, the second stimulus may be a single twitch, which is optionally applied a plurality of times at a given frequency (e.g., 20 pulses at a frequency of 1 Hz (1 stimulation/sec)). The nerve and muscle responses are recorded using the sensing electrodes. After the second stimulus is applied, the amplitudes of the muscle responses are measured. The number of second stimuli (delivered at a frequency of 1 Hz) that elicit a non-zero response are counted. As the neuromuscular block deepens, the number of second stimuli that elicit a response decreases. In other words, the PTC value decreases for deeper levels of neuromuscular block.

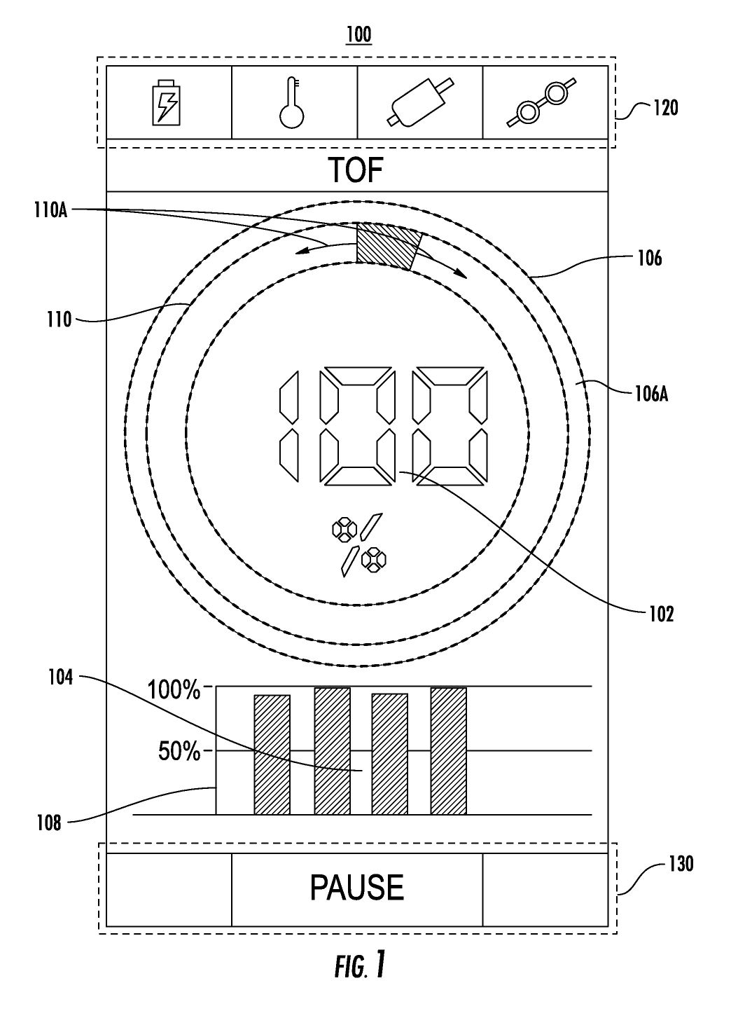

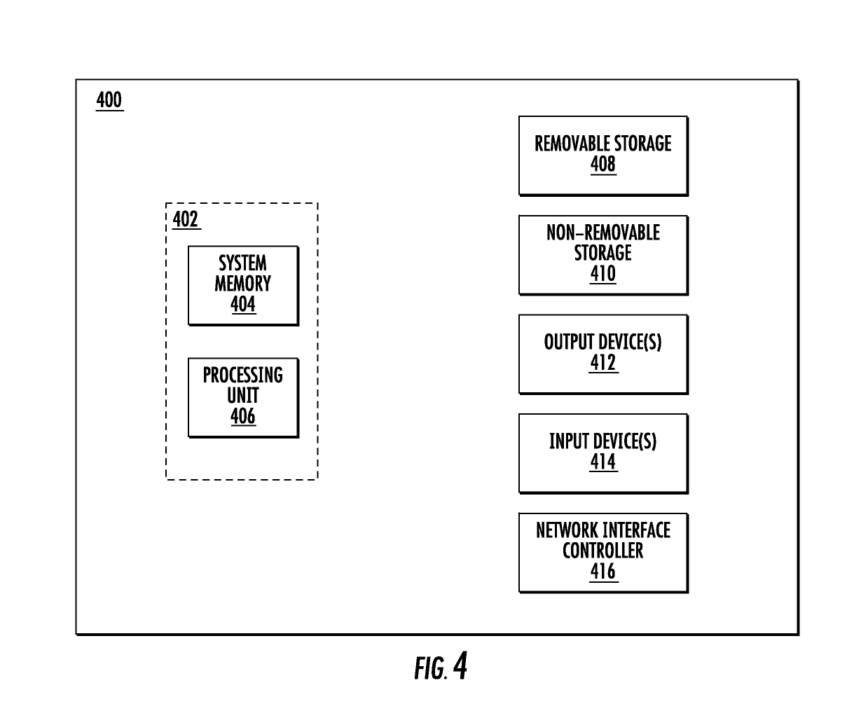

A system including a GUI for intuitively presenting the degree of neuromuscular block in the patient is discussed below. The system can optionally be the neuromuscular monitoring system discussed above. For example, a system for displaying a degree of neuromuscular block in a patient can include: a display unit having a GUI; a processor; and a memory coupled to the processor. The system can be a computing device such as the computing device discussed below with regard to FIG. 4, for example. Additionally, an example GUI 100 is shown in FIG. 1. The system can be configured to: receive data in response to a pattern of one or more stimuli applied to the patient according to a stimulation protocol; determine the degree of neuromuscular block in the patient based on the received data; display a numerical representation 102 corresponding to the degree of neuromuscular block in the patient; display a graphical representation 104 corresponding to the degree of neuromuscular block in the patient and display a timer 110 related to the stimulation protocol on the GUI. According to some implementations, the numerical representation 102 can be displayed in a first region 106 of the GUI, and the graphical representation 104 can be displayed in a second region 108 of the GUI. In some implementations, the first region 106 and the second region 108 are non-overlapping regions on the GUI. Additionally, a display color of at least a portion of the first region 106A, the numerical representation 102 and the graphical representation 104 can be configured to dynamically change based on the degree of neuromuscular block in the patient.

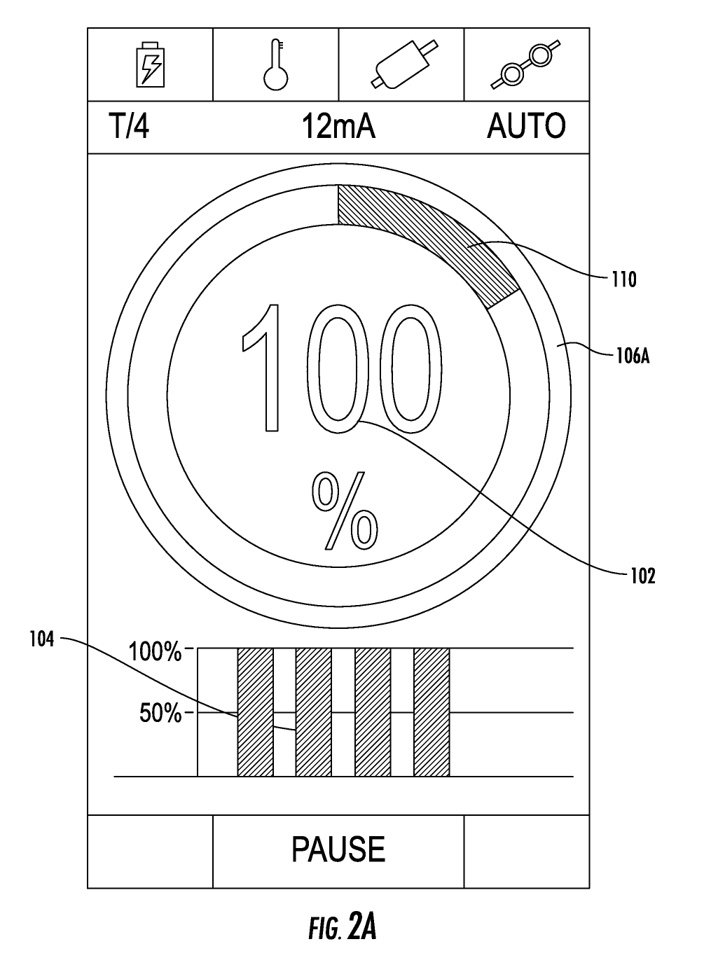

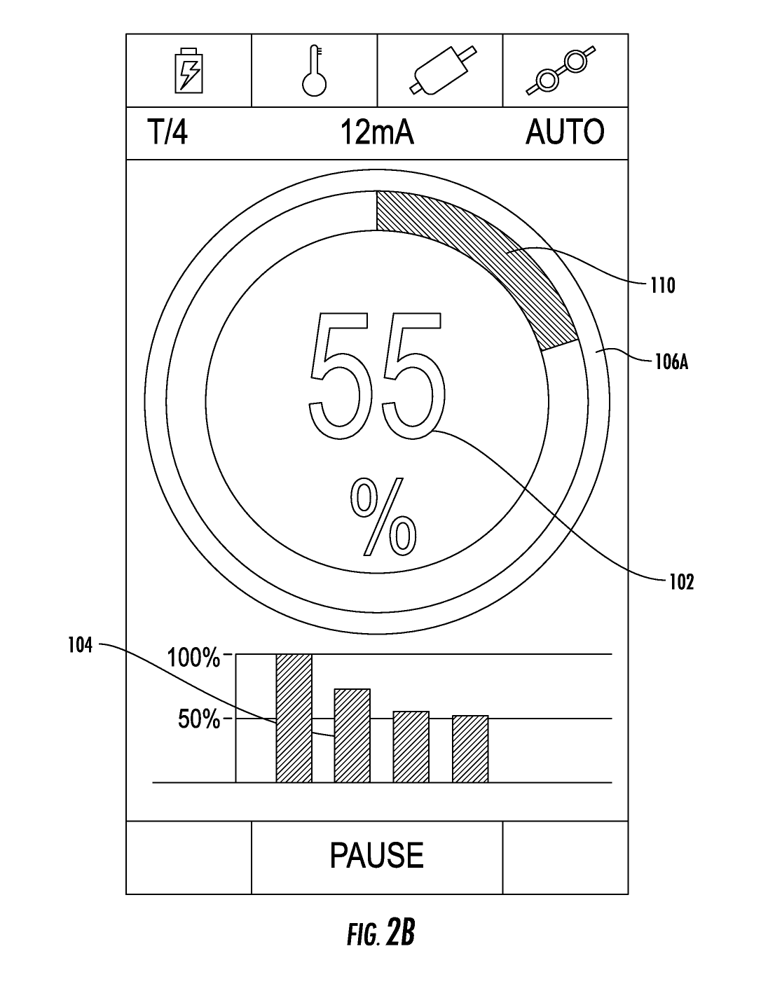

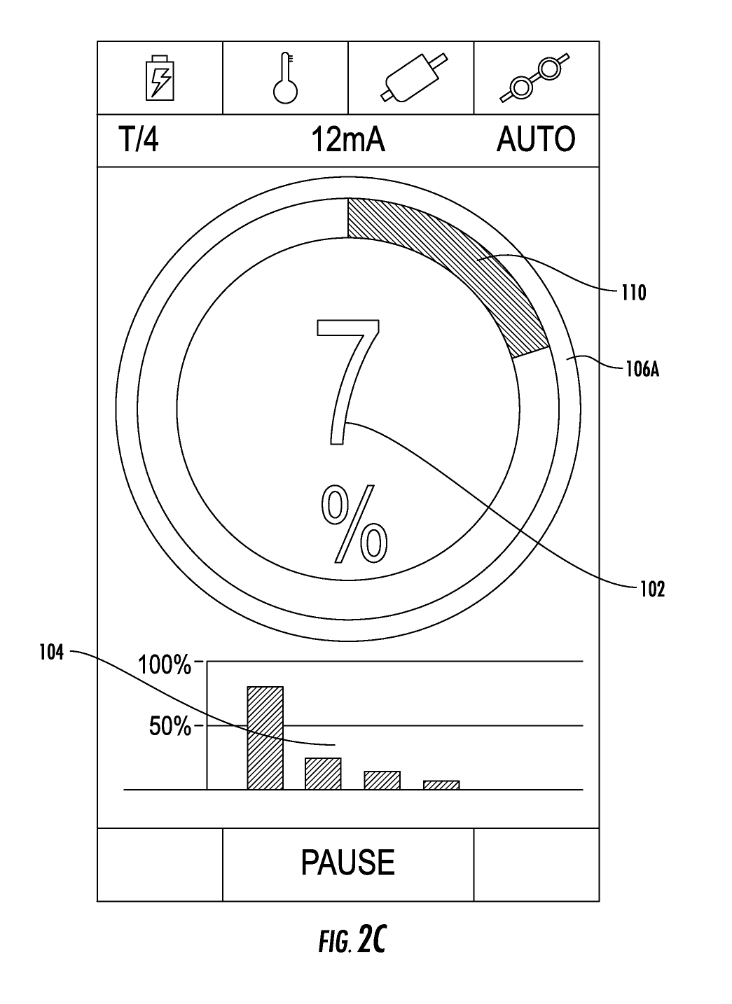

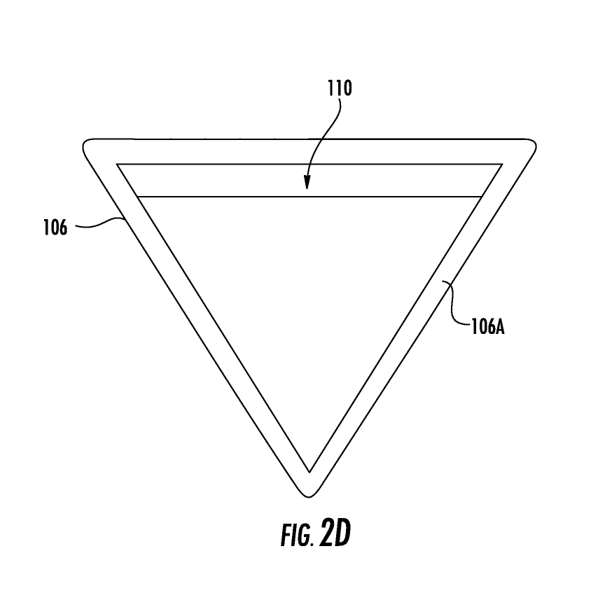

In some implementations, the numerical representation 102 can be a ratio or percentage related to the degree of neuromuscular block in the patient. Alternatively or additionally, the graphical representation 104 can depict an electrical response of a muscle to the pattern of one or more stimuli applied to the patient according to the stimulation protocol. As shown in FIGS. 2A-2C, the numerical representation 102 corresponding to the degree of neuromuscular block in the patient changes from 100% to 55% to 7%, respectively. Additionally, in FIGS. 2A-2C, the graphical representation 104 corresponding to the degree of neuromuscular block in the patient also changes. In FIGS. 2A-2C, the graphical representation 104 can be a graph such as a bar graph, for example, with a magnitude of the muscle response on one axis and time on the other axis. The magnitude of the muscle response can be a raw magnitude or a magnitude relative to a control for each successive stimulus in the pattern of stimuli. It should be understood that the graphical representation 104 in FIGS. 2A-2C depicts the fading neuromuscular response as the neuromuscular blocking agents take effect in the patient.

Additionally, the display color can be a first color when the numerical representation 102 is greater than or equal to a first predetermined value. This is shown in FIG. 2A where the display color is green. The display color can be a second color when the numerical representation 102 is greater than or equal to a second predetermined value and less than the first predetermined value. This is shown in FIG. 2B where the display color is yellow. The display color can be a third color when the numerical representation 102 is less than the second predetermined value. This is shown in FIG. 2C where the display color is red. By changing the display color of the portion of the first region 106A, the numerical representation 102 and the graphical representation 104 as the degree of neuromuscular block changes, it is possible to more intuitively display to the user of the system the change in the degree of neuromuscular block in the patient.

In some implementations, the first predetermined value can be 0.9 or 90% and the second predetermined value can be 0.4 or 40%. As discussed above, the first, second and third colors can be green, yellow and red, respectively. It should be understood, however, that this disclosure contemplates that the first and second predetermined values can have other values. Additionally, it should be understood that this disclosure contemplates that the first, second and third colors can be other colors. The first and second predetermined values and first, second and third colors discussed above are provided only as examples.

In some implementations, the first region 106 of the GUI can define a closed-loop shape, and the at least a portion of the first region 106A can extend adjacent to at least a portion of a perimeter of the closed-loop shape. As shown in FIG. 1, the at least a portion of the first region 106A is between a pair of dotted lines. For example, the closed-loop shape can be at least one of a circle or a polygon. As shown in FIGS. 1 and 2A-2C, the first region 106 of the GUI is a circle. As shown in FIG. 2D, the first region 106 of the GUI is a polygon. In particular, the first region 106 of the GUI in FIG. 2D is a triangle. As discussed below, the system can be configured to change the closed-loop shape of the first region 106 of the GUI based on the stimulation protocol, which makes it possible to more intuitively display to the user of the system the stimulation protocol being used. This disclosure contemplates that the portion of the first region 106A can extend along an entire perimeter of the closed-loop shape, which is shown in FIGS. 1 and 2A-2C. Alternatively, this disclosure contemplates that the portion of the first region 106A can extend along only a portion of the perimeter of the closed-loop shape, which is shown in FIG. 2D.

In some implementations, the timer 110 can be a graphical timer that extends adjacent to at least a portion of the perimeter of the closed-loop shape. As shown in FIG. 1, the timer 110 is between a pair of dotted lines. For example, the timer can extend adjacent to the at least a portion of the first region 106A. This disclosure contemplates that the at least a portion of the first region 106A and the timer 110 can be directly adjacent (e.g., touching) or spaced apart. Additionally, this disclosure contemplates that the timer 110 can be arranged either inside or outside a perimeter of the first region 106. Further, similarly to the at least a portion of the first region 106A, the timer 110 can extend adjacent to an entire perimeter of the first region (e.g., FIGS. 1 and 2A-2C) or only a portion of the perimeter of the first region (e.g., FIG. 2D).

The graphical timer 110 can depict a time between successive applications of the pattern of one or more stimuli applied to the patient according to the stimulation protocol, for example. For example, according to the train-of-four protocol or TOF count protocol, a period of 12 seconds elapses between applications of successive trains of stimulation pulse. According to the tetanic protocol or PTC protocol, a period of 120 seconds elapses between applications of successive tetanic stimulations. It should be understood that this disclosure should not be limited to 12 seconds and 120 seconds between successive applications of the stimulation protocols, respectively. Thus, the timer 110 can be used to depict the time between successive applications of the pattern of one or more stimuli. In some implementations, the memory can have further computer-executable instructions stored thereon that, when executed by the processor, cause the system to dynamically change the graphical timer 110 in a clockwise or counterclockwise direction. This is shown in FIG. 1 by arrows 110A. For example, a portion of the timer 110 can change color and/or change intensity to illustrate the elapse of time.

In some implementations, the pattern of one or more stimuli comprises a plurality of stimuli, each stimulus being applied after a predetermined time interval. Additionally, the memory can have further computer-executable instructions stored thereon that, when executed by the processor, cause the system to record an electrical response of a muscle to each of the plurality of stimuli.

Optionally, the graphical representation 104 can be an amplitude of the electrical response of the muscle to each of the plurality of stimuli. Alternatively or additionally, the graphical representation 104 can be a ratio of an amplitude of the electrical response of the muscle to each of the plurality of stimuli to a control amplitude. In some implementations, the control amplitude can be an amplitude of the electrical response of the muscle to one of the plurality of stimuli applied at approximately a beginning of the stimulation protocol. Optionally, the control amplitude can be an amplitude of the electrical response of the muscle to a first stimulus of the plurality of stimuli. As discussed above, FIGS. 2A-2C show that the electrical response of the muscle to one or more of the plurality of stimuli diminish as the degree of neuromuscular block increases. In particular, in FIGS. 2B and 2C, the graphical representation 104 of the degree of neuromuscular block, which are bar graphs illustrating the electrical response of the muscle to each of the plurality of stimuli, diminish for each successive stimulus in the pattern of stimuli.

In some implementations, the plurality of stimuli can include at least four stimuli. Alternatively or additionally, the stimulation protocol is a train-of-four protocol, a train-of-four count protocol, a tetanic protocol or a post-tetanic count protocol. In some implementations, the first region 106 of the GUI can define a circle when the protocol is a train-of-four protocol or a train-of-four count protocol. This is shown in FIGS. 1 and 2A-2C. In other implementations, the first region 106 of the GUI can define a polygon when the protocol is a tetanic protocol or a post-tetanic count protocol. For example, the polygon can be a triangle. This is shown in FIG. 2D.

Alternatively or additionally, the numerical representation 102 can optionally be a count of each non-zero electrical response of the muscle to the plurality of stimuli. For example, as discussed above, the count can optionally be the TOF count or the PTC count. In these implementations, the numerical representation 102 can be related to the degree of neuromuscular block in the patient.

Optionally, the memory can have further computer-executable instructions stored thereon that, when executed by the processor, cause the system to display at least one icon 120 on the GUI. For example, the icon 120 can indicate at least one of a battery charge, the patient's skin temperature or a system status. The system status can include an indication as to whether the stimulator and/or recording electrodes are connected (e.g., by measuring the impendence of the connections). In some implementations, the icon 120 can be a first color (e.g., green) when the status is positive/good, and the icon can be a second color (e.g., red) when the status is negative/bad. Additionally, audible alarms can optionally be used in conjunction with the icon 120 to provide warnings to the user of the system.

Alternatively or additionally, the memory can have further computer-executable instructions stored thereon that, when executed by the processor, cause the system to display a menu bar 130 on the GUI. The menu bar 130 can be used to allow the user to navigate system functions such as start/stop/pause the stimulation protocol, access menu options, access system settings, etc. It should be understood that the menu bar 130 can have any number of configurations and that the menu bar 130 is only provided as one example.

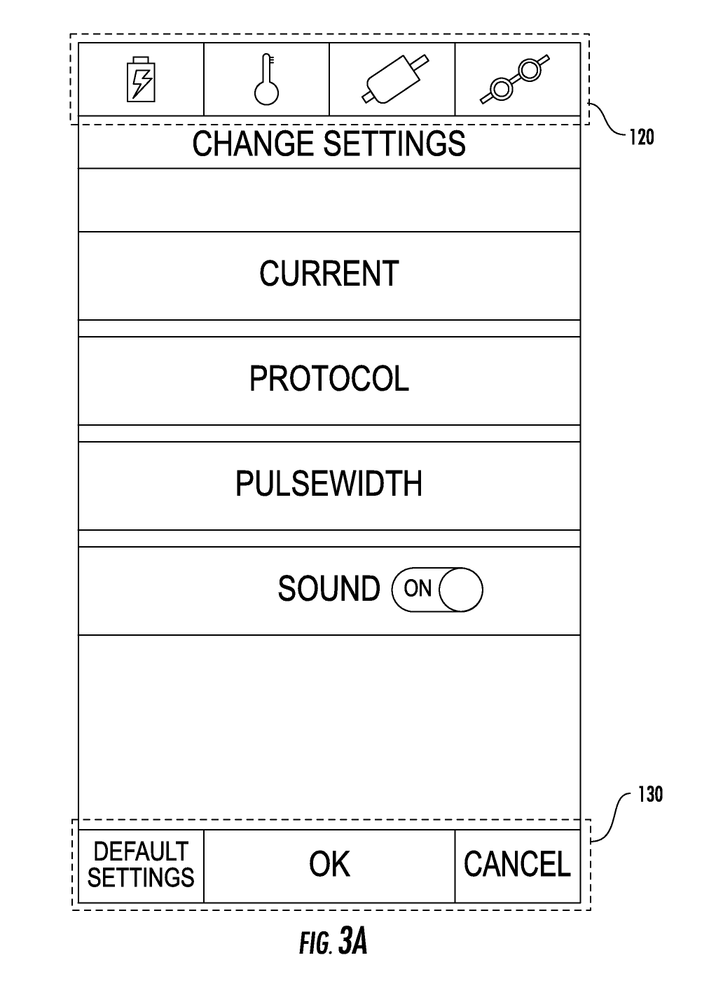

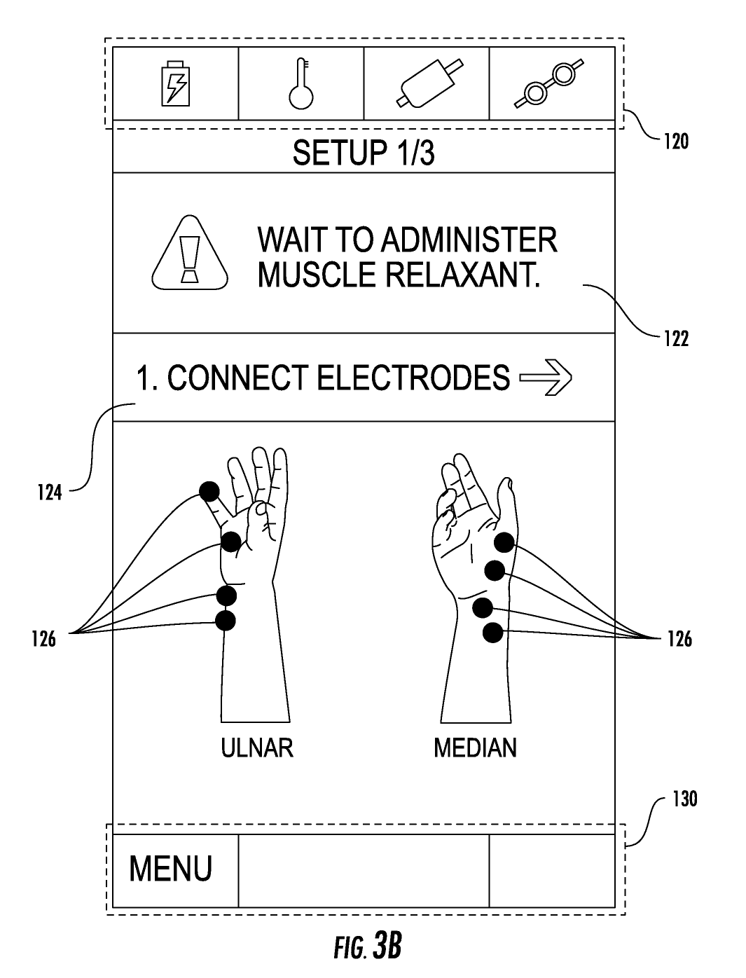

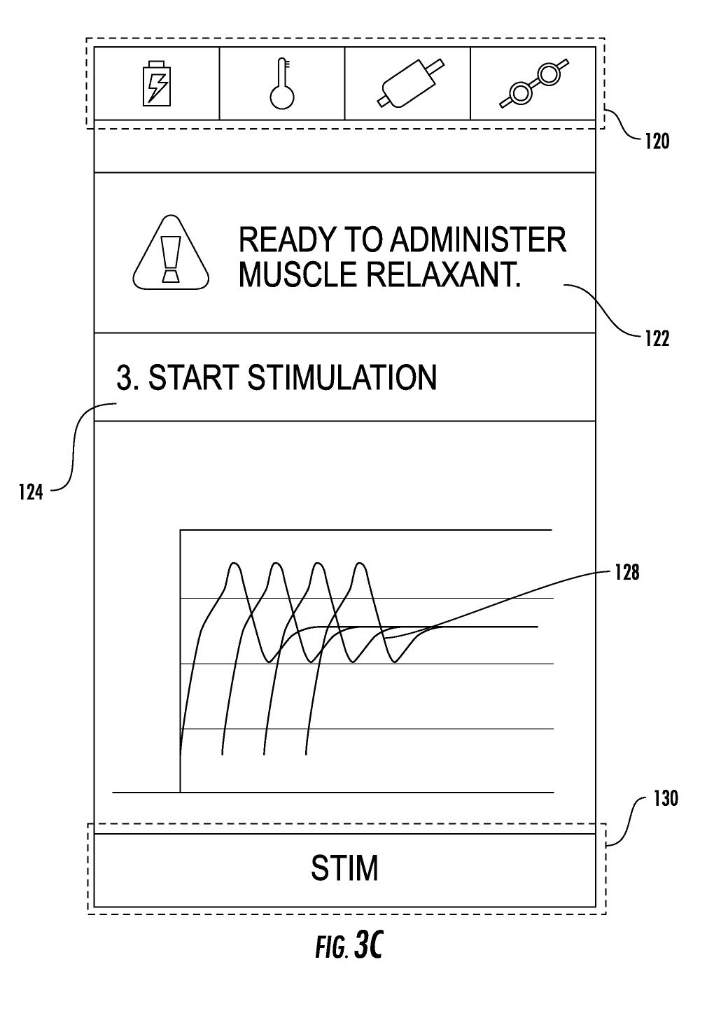

Referring now to FIGS. 3A-3C, additional example GUIs according to implementations discussed herein are shown. The GUIs shown in FIGS. 3A-3C are examples of control and/or setup GUIs. Similarly to FIGS. 1 and 2A-2C, the GUIs can optionally include the icon 120 and/or the menu bar 130. In FIG. 3A, a GUI for selecting system settings is shown. For example, the user can adjust the stimulation current (e.g., stimulation intensity). For example, the stimulation current can optionally range between 0 and 100 mA, which allows the user to identify the supra-maximal or submaximal current by adjusting the stimulation current incrementally (e.g., in 5 mA increments). The user can also select the protocol such as the single twitch, TOF, TOF count, tetanic or PTC protocol. Additionally, the user can select the pulsewidth of the stimulus, which is variable as discussed above.

In FIG. 3B, a GUI that is displayed while the user connects the stimulator and recording electrodes to the patient is shown. In particular, this GUI can be displayed while validating the stimulator and recording electrode connectivity. The GUI can optionally include a warning 122 (e.g., “WAIT TO ADMINISTER MUSCLE RELAXANT”) and an instruction 124 (“CONNECT ELECTRODES”). The warning 122 can be configured to have variable intensity and/or color in order to convey information to the user. The instruction 124 can inform the user of or allow the user to select the next step in the setup sequence. Additionally, the GUI can indicate the status of the setup sequence step. In FIG. 3B, two human hands are shown on the GUI. The status of stimulator and recording electrodes 126 are also shown relative to the human hands. The status of the stimulator and recording electrodes 126 can be updated in real-time on the GUI. For example, the color and/or intensity of the status of the stimulator and recording electrodes 126 can be configured to change based on whether the electrodes are connected (e.g., by measuring the impedance of the connections). Once the electrodes are connected, the user can select the instruction 124 to move to the next step in the sequence, for example.

In FIG. 3C, a GUI that is displayed while the system validates the nerve and/or muscle response to stimulation is shown. Similarly to above, the GUI can include optionally a warning 122 (e.g., “READY TO ADMINISTER MUSCLE RELAXANT”) and an instruction 124 (“START STIMULATION”). Before beginning the stimulation protocol, the system can validate sufficient nerve and/or muscle response. As shown in FIG. 3C, the evoked muscle response 128 to a pattern of test stimuli (e.g., according to the TOF protocol in FIG. 3C) can be displayed on the GUI. In particular, the evoked muscle response 128 shows four, well-formed muscle responses in FIG. 3C, which indicates that the user can proceed with the stimulation.

It should be appreciated that the logical operations described herein with respect to the various figures may be implemented (1) as a sequence of computer implemented acts or program modules (i.e., software) running on a computing device, (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

When the logical operations described herein are implemented in software, the process may execute on any type of computing architecture or platform. For example, referring to FIG. 4, an example computing device upon which embodiments of the invention may be implemented is illustrated. The computing device 400 may include a bus or other communication mechanism for communicating information among various components of the computing device 400. In its most basic configuration, computing device 400 typically includes at least one processing unit 406 and system memory 404. Depending on the exact configuration and type of computing device, system memory 404 may be volatile (such as random access memory (RAM)), non-volatile (such as read-only memory (ROM), flash memory, etc.), or some combination of the two. This most basic configuration is illustrated in FIG. 4 by dashed line 402. The processing unit 406 may be a standard programmable processor that performs arithmetic and logic operations necessary for operation of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display unit having a GUI, speakers, printer, etc. may also be included. The additional devices may be connected to the bus in order to facilitate communication of data among the components of the computing device 400. All these devices are well known in the art and need not be discussed at length here.

The processing unit 406 may be configured to execute program code encoded in tangible, computer-readable media. Computer-readable media refers to any media that is capable of providing data that causes the computing device 400 (i.e., a machine) to operate in a particular fashion. Various computer-readable media may be utilized to provide instructions to the processing unit 406 for execution. Common forms of computer-readable media include, for example, magnetic media, optical media, physical media, memory chips or cartridges, a carrier wave, or any other medium from which a computer can read. Example computer-readable media may include, but is not limited to, volatile media, non-volatile media and transmission media. Volatile and non-volatile media may be implemented in any method or technology for storage of information such as computer readable instructions, data structures, program modules or other data and common forms are discussed in detail below. Transmission media may include coaxial cables, copper wires and/or fiber optic cables, as well as acoustic or light waves, such as those generated during radio-wave and infra-red data communication. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

Computing device 400 typically includes a variety of computer-readable media. Computer-readable media can be any available media that can be accessed by device 400 and includes both volatile and non-volatile media, removable and non-removable media. Computer storage media include volatile and non-volatile, and removable and non-removable media implemented in any method or technology for storage of information such as computer readable instructions, data structures, program modules or other data. System memory 404, removable storage 408, and non-removable storage 410 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 400. Any such computer storage media may be part of computing device 400.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A system for displaying a degree of neuromuscular block in a patient, comprising:
a display unit having a graphical user interface (GUI);
a processor; and
a memory coupled to the processor, the memory having computer-executable instructions stored thereon that, when executed by the processor, cause the system to:
receive data in response to a pattern of one or more stimuli applied to the patient according to a stimulation protocol;
determine the degree of neuromuscular block in the patient based on the received data;
display a numerical representation corresponding to the degree of neuromuscular block in the patient, the numerical representation being displayed in a first region of the GUI;
display a graphical representation corresponding to the degree of neuromuscular block in the patient, the graphical representation being displayed in a second region of the GUI; and
display a timer related to the stimulation protocol on the GUI.

2-5. (canceled)

6. The system of claim 1, wherein the graphical representation depicts an electrical response of a muscle to the pattern of one or more stimuli applied to the patient according to the stimulation protocol.

7. The system of claim 1, wherein the first region of the GUI defines a closed-loop shape, and the at least a portion of the first region extends adjacent to at least a portion of a perimeter of the closed-loop shape.

8. (canceled)

9. The system of claim 7, wherein the timer is a graphical timer that extends adjacent to at least a portion of the perimeter of the closed-loop shape.

10. The system of claim 9, wherein the graphical timer depicts a time between successive applications of the pattern of one or more stimuli applied to the patient according to the stimulation protocol.

11. (canceled)

12. The system of claim 1, wherein the pattern of one or more stimuli comprises a plurality of stimuli, each stimulus being applied after a predetermined time interval, wherein the memory has further computer-executable instructions stored thereon that, when executed by the processor, cause the system to record an electrical response of a muscle to each of the plurality of stimuli.

13. The system of claim 12, wherein the graphical representation comprises an amplitude of the electrical response of the muscle to each of the plurality of stimuli.

14. (canceled)

15. The system of claim 12, wherein the graphical representation comprises a ratio of an amplitude of the electrical response of the muscle to each of the plurality of stimuli to a control amplitude.

16. The system of claim 12, wherein the graphical representation comprises a ratio of an amplitude of the electrical response of the muscle to each of the plurality of stimuli to a control amplitude, and wherein the control amplitude is an amplitude of the electrical response of the muscle to one of the plurality of stimuli applied at approximately a beginning of the stimulation protocol.

17. (canceled)

18. The system of claim 12, wherein the plurality of stimuli comprises at least four stimuli.

19. The system of claim 18, wherein the graphical representation comprises a ratio of an amplitude of each of a plurality of subsequently applied stimuli of the plurality of stimuli to an amplitude of the electrical response of the muscle to a prior stimulus of the plurality of stimuli.

20. The system of claim 18, wherein the graphical representation comprises a ratio of an amplitude of the electrical response of the muscle to a second, third and fourth stimulus of the plurality of stimuli to an amplitude of the electrical response of the muscle to a first stimulus of the plurality of stimuli.

21. (canceled)

22. The system of claim 12, wherein the numerical representation is a count of each non-zero electrical response of the muscle to the plurality of stimuli, the numerical representation being related to the degree of neuromuscular block in the patient.

23. The system of claim 1, wherein the stimulation protocol is at least one of a train-of-four protocol, a train-of-four count protocol, a tetanic protocol or post-tetanic count protocol.

24-29. (canceled)

30. The system of claim 1, wherein the memory has further computer-executable instructions stored thereon that, when executed by the processor, cause the system to display at least one icon or a menu bar on the GUI.

31. (canceled)

32. (canceled)

33. The system of claim 1, wherein a display color of at least a portion of the first region, the numerical representation and the graphical representation is configured to dynamically change based on the degree of neuromuscular block in the patient.

34. The system of claim 33, wherein the numerical representation is a ratio, a percentage or a count of each non-zero electrical response of a muscle to the pattern of one or more stimuli, the ratio, percentage or the count being related to the degree of neuromuscular block in the patient.

35. The system of claim 34, wherein the display color is a first color when the numerical representation is greater than or equal to a first predetermined value, the display color is a second color when the numerical representation is greater than or equal to a second predetermined value and less than the first predetermined value and the display color is a third color when the numerical representation is less than the second predetermined value.

36. The system of claim 35, wherein the first predetermined value is 0.9 or 90% and the second predetermined value is 0.4 or 40%.

37. A system for intuitively displaying a degree of neuromuscular block in a patient, comprising:
a display unit having a graphical user interface (GUI);
a processor; and
a memory coupled to the processor, the memory having computer-executable instructions stored thereon that, when executed by the processor, cause the system to:
display a numerical representation corresponding to the degree of neuromuscular block in the patient;
display a graphical representation corresponding to the degree of neuromuscular block in the patient; and
display a dynamic graphical timer on the GUI, wherein the dynamic graphical timer is related to a selected stimulation protocol.

38. The system of claim 37, wherein the dynamic graphical timer depicts a time between successive applications of stimuli according to the selected stimulation protocol.

39. The system of claim 37, wherein the numerical representation is displayed in a first region of the GUI, and the graphical representation is displayed in a second region of the GUI, the first and second regions being non-overlapping regions of the GUI.

40. The system of claim 39, wherein the first region defines a closed-loop shape, and wherein the memory has further computer-executable instructions stored thereon that, when executed by the processor, cause the system to change the closed-loop shape based on the selected stimulation protocol.

41. The system of claim 39, wherein a display color of at least a portion of the first region, the numerical representation and the graphical representation is configured to dynamically change based on the degree of neuromuscular block in the patient.
